(19) United States
(12) Patent Application Publication
(10) Pub. No.: US 2005/0287180 A1
(43) Pub. Date: Dec. 29, 2005

(54) Phospholipid compositions and methods for their preparation and use

(76) Inventor: Andrew Xian Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 11/152,855

(22) Filed: Jun. 14, 2005

Related U.S. Application Data
(60) Provisional application No. 60/580,183, filed on Jun. 15, 2004.

(51) Int. Cl. A61K 45/00; A61K 38/18; A61K 9/127

(52) U.S. Cl. 424/400; 424/93.7; 514/2

(57) ABSTRACT

The present invention provides compositions that comprise a phospholipid component (that contains one or more phospholipids) and a pharmaceutically acceptable fluid carrier, where the phospholipid component is in the range from about 10% to about 90% of the total weight. Optionally, the compositions may further comprise non-phospholipid filler materials, where the amount of the non-phospholipid filler materials is in the range from about 5% to about 50% of the total weight. In certain embodiments, the compositions may be injectable, non-liposomal, and/or in form of a gel or a paste. The compositions of the present invention are useful for repairing and augmenting soft and/or hard tissues or for sustained local drug delivery.

### CROSS-REFERENCE TO RELATED APPLICATION

This application claims the benefit of U.S. Provisional Patent Application No. 60/580,183 filed Jun. 15, 2004, which is incorporated herein by reference in its entirety.

### BACKGROUND OF THE INVENTION

1. Field of the Invention

The present invention relates generally to the preparation and use of biocompatible implant compositions. More particularly, the present invention relates to phospholipid compositions for soft and hard tissue repair and augmentation and for sustained local drug delivery.

2. Description of the Related Art

Implant compositions for soft and hard tissue repair and augmentation consist of primarily collagen and hyaluronic acid. The collagen and hyaluronic acid implant products marketed in the United States include CosmoDerm™, CosmoPlast™, Zyderm® and Zyplast® and Hylaform®.

Collagen and hyaluronic acid compositions have been used primarily for superficial soft tissue augmentation, i.e., near the surface of the skin as a dermal filler for the removal or improvement of scars caused by acne, correction of facial (wrinkle) lines, and enhancement or filling in of certain specific facial features such as the lips or chin.

The use of collagen or hyaluronic acid as the primary matrix material in soft and hard tissue implant compositions has several limitations. The preparation of collagen suitable for human use is relatively time consuming and expensive. In particular, the complete removal of contaminating and potentially immunogenic substances to produce “atelocollagen” is a relatively complex and expensive procedure. The emergence of mad cow disease (bovine spongiform encephalopathy or BSE) has severely limited safe sources of bovine collagens available for human use.

As dermal fillers, collagen implants tend to be insufficient in their persistence, shape retention and toughness. For example, the wrinkle-removal effect of a fibrillar collagen implant typically lasts for only 1-6 months, thus requiring repeated injections

Another dermal filler material, hyaluronic acid, suffers from the similar limitations; hyaluronic acid implantation is not permanent. Hyaluronic acid, natural or synthetic once injected into the skin will gradually break down and be absorbed by the body. In most cases, the augmentation usually lasts anywhere between 1-5 months. To maintain the initial results, repeated treatments or top-up treatments will be necessary, usually 2 to 3 treatments per year.

Collagen or hyaluronic acid is also used as a carrier vehicle for other solid bulking agents or dermal fillers such as hydroxyapatite, microspheres of polymethylmethacrylate (PMMA, a non-reabsorbable polymer) or poly lactide-co-glycolide (PLGA, a reabsorbable polymer) or ceramic materials. An ideal carrier vehicle should provide a sustained support of microspheres in order to allow for tissue ingrowth to fill the space between the microspheres. However, due to its short persistence time, a collagen or hyaluronic acid carrier vehicle usually disappears before the ingrowth of tissue takes place, resulting in collapse of the microspheres.

PMMA as a permanent bulking material is available as microspheres (Artifill™ by Artes Medical, Inc.), and PMMA microspheres must be suspended in a collagen vehicle for injection. The collagen-suspended PMMA microsphere injectable implant product therefore suffers from the same limitations as the collagen only dermal filler products. Moreover, fibrillar collagen requires storage in a refrigerator.

Accordingly, there is a need for improved implant materials for soft and hard tissue repair and augmentation. The present invention fulfills such a need and provides other related advantages.

### BRIEF SUMMARY OF THE INVENTION

The present invention provides phospholipid compositions and methods of making and using such compositions. More specifically, in one aspect, the present invention provides a composition adapted for use as a tissue filler that comprises a phospholipid component and a pharmaceutically acceptable fluid carrier. The phospholipid component may contain one or more phospholipids, and is in the range from about 10% to about 90% of the total weight of the composition. In certain embodiments, the composition is injectable, non-liposomal, and/or in form of a gel or a paste.

The phospholipid compositions are generally processed to minimize immune and inflammatory response, and are present in a pharmaceutically acceptable fluid carrier, typically an aqueous media or pharmaceutically acceptable organic vehicle composition.

The use of biocompatible phospholipid compositions (e.g., phospholipid pastes) as a primary implant component is advantageous in a number of respects. The phospholipid compositions (e.g., phospholipid pastes) are able to become anchored within a host's own tissue, resulting in a very persistent implant which remains stable over extended time periods. Despite this ability to interact with the host tissue, the phospholipid compositions are substantially immunologically inert and cause little or no immune or inflammatory response. Additionally, the phospholipid compositions are inexpensive relative to other implant matrix materials, such as collagen or hyaluronic acid, thus reducing the cost of the compositions of the present invention. Moreover, by employing phospholipids as a filler material, soft tissue implants having a wider range of consistency or firmness can be achieved than with either the hyaluronic acid or collagen implant. Surprisingly, these benefits are achieved by varying the type and concentration of phospholipids.

The compositions of the present invention may further comprise a non-phospholipid filler material, where the ratio of the phospholipid to the non-phospholipid filler materials is selected to provide for a desired consistency, firmness, persistence, and injectability in certain embodiments, in the resulting implant.

For instance, by combining with a resorbable or inert (non-resorbable) non-phospholipid filler material, such as microspheres of PMMA, PLGA or hydroxyapatite, the long-term persistence of the implant can be programmed depending on the particular application, whereby the phospholipid component of an implant composition provides supporting matrix to suspend the microspheres for sufficiently long time to allow for tissue ingrowth between the microspheres.

The compositions of the present invention may further comprise one or more biologically active agents, including but without limitation, gene transfer vectors, local anesthetics, anti-inflammatory agents, anti-cancer agents, anti-infectious agents, hormones, bone metabolism regulators, anti-convulsants, anti-depressants, analgesics, antipsychotic agents, anti-diabetic agents, anti-parkinisonian agents, smoking cessation aids, urinary tract agents, anti-osteoporosis agents, anti-obesity agents, cardiotonic agents, fertility agents, contraceptives, preservatives, and cell adhesion promoters.

In a related aspect, the present invention provides a composition adapted for sustained local drug delivery that comprises a phospholipid component, a pharmaceutically acceptable fluid carrier, and a biologically active agent. The phospholipid component may contain one or more phospholipids and is in the range from about 10% to about 90% of the total weight of the composition. The biologically active agent is in a pharmaceutically effective concentration. In certain embodiments, the composition is injectable, non-liposomal, and/or in form of a gel or a paste, where the phospholipid components affect the release rate and duration of the biologically active agent. In certain embodiments, the composition adapted for sustained local drug delivery further comprises a non-phospholipid filler component where both the phospholipid component and the non-phospholipid filler component affect the release rate and duration of the biologically active agent. Typically, the biologically active agent is released at a pharmaceutically effective amount from the composition to the site where the composition is administered for at least one week.

In another aspect, the present invention further provides methods for preparing phospholipid compositions described herein (including those adapted for use as a tissue filler and those for sustained local drug delivery), where the phospholipid component is suspended in a fluid carrier and optionally with non-phospholipid filler materials such as microspheres of PMMA, PLGA or hydroxyapatite. In certain embodiments, such methods comprise homogenizing (e.g., mechanically agitating) the phospholipid compositions to produce injectable materials. In certain embodiments, the resulting phospholipid compositions are non-liposomal and/or in form of a gel or a paste. In certain embodiments, the methods for preparing phospholipid compositions further comprising sterilizing the compositions by filtration, heat, radiation, electron beam, a combination thereof, or the like.

In yet another aspect, the present invention provides methods for using such compositions in tissue repair or augmentation or in local drug delivery. For instance, the present invention provides methods for repairing or augmenting hard tissue (e.g., bone, cartilage, and connective tissue) that comprise administering the phospholipid compositions described herein. The present invention also provides methods for dermal (including cosmetic) augmentation that comprise administering the phospholipid compositions described herein. The present invention further provides methods for tissue bulking (e.g., bulking the vocal cord, the lower esophageal sphincter, the diaphragm, the bladder sphincter, or the ureathra) in a mammal that comprise administering the phospholipid compositions to a site in need thereof.

In certain embodiments, the administration may be performed using a needle having a diameter of 21 gauge or higher. Such administration is particularly useful for deep tissue injection to locations near bone and cartilage for purposes such as sphincter repair, nasal repair, and the like.

In another aspect, the present invention provides a method for local delivery of a biologically active agent. In a related aspect, the present invention provides a method for treating a solid tumor comprising injecting into the solid tumor a composition that comprises a phospholipid component, a pharmaceutical acceptable fluid carrier, and an anti-tumor agent. In one embodiment, the phospholipid component is in the range from about 10% to about 90% of the total weight of the composition, and the anti-tumor agent is in a pharmaceutically effective concentration. In another related aspect, the present invention provides a method for treating chronic pain comprising administering at the site of chronic pain a composition that comprises a phospholipid component, a pharmaceutical acceptable fluid carrier, and a local anesthetic. In one embodiment, the phospholipid component is in the range from about 10% to about 90% of the total weight of the composition, and the local anesthetic is in a pharmaceutically effective concentration. In another related aspect, the present invention provides a method for treating chronic periodontal disease comprising administering at the site of chronic periodontal disease a composition that comprises a phospholipid component, a pharmaceutical acceptable fluid carrier, and an anti-infectious agent. In one embodiment, the phospholipid component is in the range from about 10% to about 90% of the total weight of the composition, and the anti-infectious agent is in a pharmaceutically effective concentration.

In certain embodiments of each method for using the phospholipid compositions described above, the compositions are injectable, non-liposomal, and/or in form of a gel or a paste.

In another aspect, the present invention provides kits for preparing and/or using a composition adapted for implantation into an animal. In certain embodiments, the kits comprise the phospholipid compositions as described herein and instructions for using the compositions. In other embodiments, the kits comprise one or more individual components of the phospholipid compositions that are packaged separately and instructions for preparing and/or using the compositions.

### BRIEF DESCRIPTION OF THE DRAWING

FIG. 1 shows the structure of phosphotidylcholine.

### DETAILED DESCRIPTION OF THE INVENTION

In one aspect, the present invention provides phospholipid compositions useful for repairing or augmenting tissues or for sustained local drug delivery. Phospholipid compositions according to the present invention comprise a phospholipid component and a pharmaceutically acceptable fluid carrier, wherein the phospholipid component is in the range from about 10% to about 90% of the total weight. Optionally, a non-phospholipid filler component and/or pharmaceutically active component(s) may be combined as part of the phospholipid compositions.

The compositions of the present invention possess one or more of the following desirable characteristics: (1) biocompatible (i.e., substantially non-toxic), (2) non-allergenic (i.e., produce no or tolerable levels of immune and inflammatory responses), (3) of non-animal origin, (4) stable at room temperature, (5) readily syringeable and/or injectable so that they can be introduced to a desired soft tissue site using a catheter or a fine gauge needle, (6) persistent at the site of administration, preferably adhering to the soft tissue into which they have been administered, (7) tough and elastic (i.e. capable of bearing loads without undergoing excessive or permanent deformation), (8) intrudable (i.e., form a relatively dispersed, irregularly shaped mass within the tissue where the composition has been introduced), (9) bio-absorbable, and (10) capable of providing sustained local drug delivery.

In certain embodiments, the phospholipid compositions of the present invention comprise a phospholipid component present in a pharmaceutically acceptable fluid carrier to form a solution or dispersion.

By “solution” it is meant a clear liquid in which a solute is completely dissolved in a solvent to form a molecularly dispersed system. The solute of this invention is primarily a phospholipid component and solvent is a pharmaceutically acceptable fluid carrier.

By “dispersion” it is meant that a combination of the phospholipid component and optionally a non-phospholipid filler component and the pharmaceutically acceptable fluid carrier is present as an emulsion, a suspension, a gel (hydrogel or an organogel), a paste or the mixtures thereof, in particular, a gel or a paste.

By “emulsion” it is meant a liquid mixture containing droplets of one liquid (the discrete phase) dispersed in another immiscible liquid (the continous phase). The emulsion of this invention may be either the oil-in-water or water-in-oil type or mixtures thereof. The phospholipid components may be contained in either liquid phase or both.

By “suspension” it is meant a mixture of a relatively thin consistency, comprising a solid-in-liquid mixture, wherein the solid content is up to 10% of the total weight and wherein the liquid is a pharmaceutically acceptable fluid carrier. The solid phase of a suspension of this invention is primarily a phospholipid component and optionally a non-phospholipid filler component.

By “gel” it is meant a clear or translucent and uniform colloidal mixture of a soft and malleable consistency, in a more solid form than a solution, consisting of a solid component dissolved in a dispersion medium. The solid component for preparing a gel of this invention is primarily a phospholipid component and dispersion medium is a pharmaceutically acceptable fluid carrier.

By “hydrogel” it is meant a gel wherein the dispersion medium is primarily water.

By “organogel” it is meant a gel wherein the dispersion medium is primarily a non-aqueous pharmaceutically acceptable fluid carrier.

By “paste” it is meant an opaque mixture of soft and malleable consistency, comprising a solid-in-liquid suspension of a high solid content wherein the solid content exceeds 10% of the total weight and wherein the liquid is a pharmaceutically acceptable fluid carrier. The solid phase of a paste of this invention is primarily a phospholipid component and optionally a non-phospholipid filler component and the liquid phase is the aqueous or non-aqueous pharmaceutically acceptable fluid carrier.

By “syringeable” it is meant that in certain embodiments, the compositions of the present invention may be administered with a syringe or a catheter.

By “injectable” it is meant that in certain embodiments, the composition of this invention may be administered by injection, for example, through a 21 gauge or higher needle.

The term “phospholipid component” refers to phospholipid molecules in a composition. Such molecules may be identical to, or different from, each other. In other words, a phospholipid component may comprise molecules from a single species of phospholipid, or comprise a mixture of two or more different species of phospholipids.

The term “phospholipids” refers to lipid molecules containing one or more phosphate groups, including those derived from either glycerol (phosphoglycerides, glycerophospholipids) or sphingosine (sphingolipids). They include polar lipids, and certain phospholipids that are of great importance for the structure and function of cell membranes and are the most abundant of membrane lipids. In certain embodiments, phospholipids are triglyceride derivatives in which one fatty acid has been replaced by a phospharylated group and one of several nitrogen-containing molecules. The fatty acid chains are hydrophobic (as in all fats). However, the charges on the phosphorylated and amino groups make that portion of the molecule hydrophilic. The result is an amphiphilic molecule.

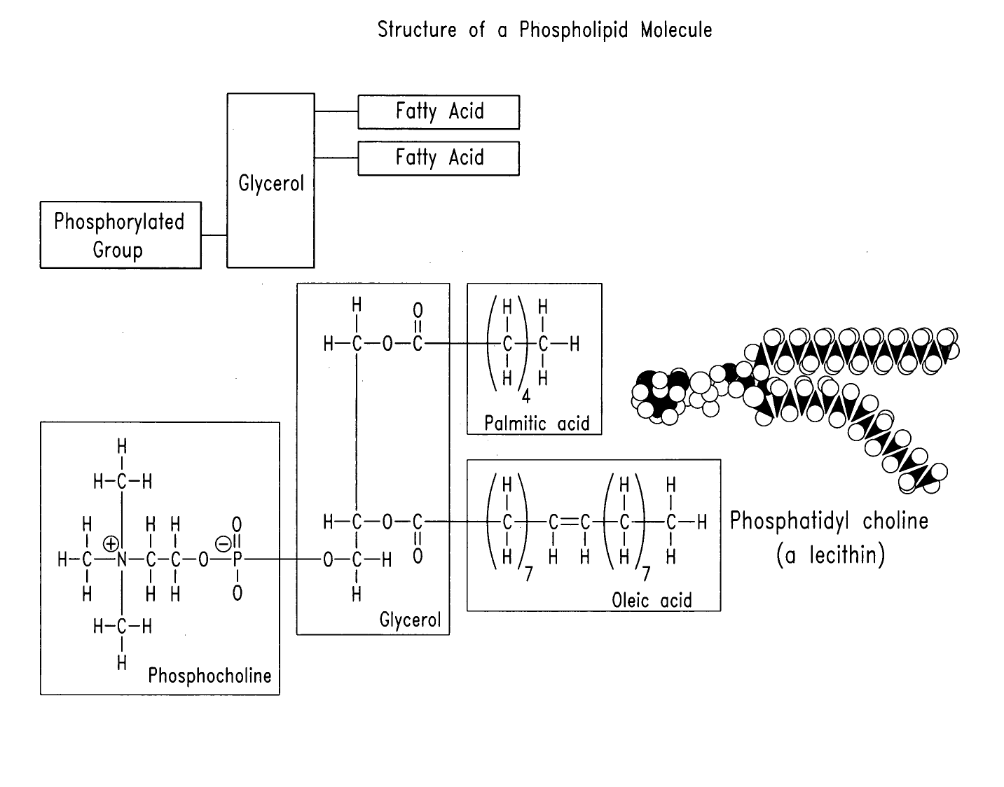

Amphiphilic phospholipids are major constituents of cell membranes. These molecules form a phospholipid bilayer with their hydrophilic (polar) heads facing their aqueous surroundings (e.g., the cytosol) and their hydrophobic tails facing each other. The most abundant and structurally most important phospholipid is phosphatidylcholine (FIG. 1).

Phospholipids are available from naturally occurring sources or by organic synthesis. Lecithin is a naturally occurring mixture of the diglycerides of stearic, palmitic, and oleic acids, linked to the choline ester of phosphoric acid, commonly called phosphatidylcholine. Hydrogenated lecithin is the product of controlled hydrogenation of lecithin.

According to the United State Pharmacopoeia (USP), lecithin is a non-proprietary name describing a complex mixture of acetone-insoluble phospholipids, which consists chiefly of phosphotidylcholine, phosphotidylethanolamine, phosphotidylserine and phosphotidylinositol, combined with various amounts of other substances such as triglycerides, fatty acids, and carbohydrates. The composition of lecithin and hence its physical properties vary depending upon the source of the lecithin and phospholipid composition, e.g., phosphotidylcholine content, etc.

The commercially available lecithin products have two primary sources: egg yolk and soybean. The CAS Registry Numbers for lecithins are as follow:
Lecithin (general): CAS 8002-43-5
Soybean lecithin or soy lecithin: CAS 8030-76-0
Egg yolk lecithin or egg lecithin: CAS 93685-90-6

Lecithin is a component in cell membranes and is therefore consumed as a normal part of the diet. It is highly biocompatible and virtually nontoxic in acute oral studies, short-term oral studies, and subchronic dermal studies in animals. Lecithin is not a reproductive toxicant, nor is it mutagenic in several assays. In a subcutaneous carcinogenicity study, no neoplasms were found in mice and rats exposed to lecithin. Lecithin and hydrogenated lecithin are generally nonirritating and nonsensitizing in animal and human skin cosmetics (Fiume Z, Final report on the safety assessment of Lecithin and Hydrogenated Lecithin, Int J Toxicol. 2001; 20 Suppl 1:21-45).

Pharmaceutically, lecithins are mainly used as dispersing, emulsifying, and stabilizing agents and are included in intramuscular (IM) and intravenous (IV) injections, parenteral nutritional formulations and topical products. Lecithin is also listed in the FDA Inactive Ingredients Guide for use in inhalations, IM and IV injections, oral capsules, suspensions and tablets, rectal, topical, and vaginal preparations.

Cosmetically, lecithin and hydrogenated lecithin are safe as used in rinse-off cosmetic products; they may be safely used in leave-on products at concentrations up to 15%, the highest concentration tested in clinical irritation and sensitization studies cosmetics.

The lecithin products preferred in some embodiments for this invention are the pharmaceutical grade lecithin products derived from soy bean, which have been used in parenteral products and are substantially free from irritating, allergenic, inflammatory agents or agents that cause other deleterious biological reactions.

Other examples of phospholipids from naturally occurring sources that may be used for this invention include sphingolipids in the form of sphingosine and derivatives (from soybean, egg, brain & milk), gangliosides, phytosphingosine and derivatives (from yeast), phosphotidylethanolamine, phosphotidylserine, and phosphotydylinositol.

Phospholipids can also be synthesized and the common synthetic phospholipids are listed below:

Diacylglycerols

1,2-Dilauroyl-sn-glycerol (DLG)
1,2-Dimyristoyl-sn-glycerol (DMG)
1,2-Dipalmitoyl-sn-glycerol (DPG)
1,2-Distearoyl-sn-glycerol (DSG)
   Phosphatidic Acids
1,2-Dimyristoyl-sn-glycero-3-phosphatidic acid, sodium salt (DMPA,Na)
1,2-Dipalmitoyl-sn-glycero-3-phosphatidic acid, sodium salt (DPPA,Na)
1,2-Distearoyl-sn-glycero-3-phosphatidic acid, sodium salt (DSPA,Na)
   Phosphocholines
1,2-Dilauroyl-sn-glycero-3-phosphocholine (DLPC)
1,2-Dimyristoyl-sn-glycero-3-phosphocholine (DMPC)
1,2-Dipalmitoyl-sn-glycero-3-phosphocholine (DPPC)
1,2-Dipalmitoyl-sn-glycero-3-phosphocholine (DPPC)
1,2-Distearoyl-sn-glycero-3-phosphocholine (DSPC)
1,2-Distearoyl-sn-glycero-3-phosphocholine (DSPC)
   Phosphoethanolamines
1,2-Dilauroyl-sn-glycero-3-phosphoethanolamine (DLPE)
1,2-Dimyristoyl-sn-glycero-3-phosphoethanolamine (DMPE)
1,2-Dipalmitoyl-sn-glycero-3-phosphoethanolamine (DPPE)
1,2-Distearoyl-sn-glycero-3-phosphoethanolamine (DSPE)
   Phosphoglycerols
1,2-Dilauroyl-sn-glycero-3-phosphoglycerol, sodium salt (DLPG)
1,2-Dimyristoyl-sn-glycero-3-phosphoglycerol, sodium salt (DMPG)
1,2-Dimyristoyl-sn-glycero-3-phospho-sn-1-glycerol, ammonium salt (DMP-sn-1-G,NH₄)
1,2-Dipalmitoyl-sn-glycero-3-phosphoglycerol, sodium salt (DPPG,Na)
1,2-Distearoyl-sn-glycero-3-phosphoglycerol, sodium salt (DSPG,Na)
1,2-Distearoyl-sn-glycero-3-phospho-sn-1-glycerol, sodium salt (DSP-sn-1G,Na)
   Phosphoserines
1,2-Dipalmitoyl-sn-glycero-3-phospho-L-serine, sodium salt (DPPS,Na)
   Mixed Chain Phospholipids
1-Palmitoyl-2-oleoyl-sn-glycero-3-phosphocholine (POPC)
1-Palmitoyl-2-oleoyl-sn-glycero-3-phosphoglycerol, sodium salt (POPG,Na)
1-Palmitoyl-2-oleoyl-sn-glycero-3-phosphoglycerol, ammonium salt (POPG,NH₄)
   Lysophospholipids
1-Palmitoyl-2-lyso-sn-glycero-0.3-phosphocholine (P-lyso-PC)
1-Stearoyl-2-lyso-sn-glycero-3-phosphocholine (S-lyso-PC)
   Pegylated Phospholipids
N-(Carbonyl-methoxypolyethyleneglycol 2000)-MPEG-2000-DPPE
1,2-dipalmitoyl-sn-glycero-3-phosphoethanolamine, sodium salt
N-(Carbonyl-methoxypolyethyleneglycol 5000)-MPEG-5000-DSPE
1,2-distearoyl-sn-glycero-3-phosphoethanolamine, sodium salt
N-(Carbonyl-methoxypolyethyleneglycol 5000)-MPEG-5000-DPPE
1,2-dipalmitoyl-sn-glycero-3-phosphoethanolamine, sodium salt
N-(Carbonyl-methoxypolyethyleneglycol 750)-MPEG-750-DSPE
1,2-distearoyl-sn-glycero-3-phosphoethanolamine, sodium salt
N-(Carbonyl-methoxypolyethyleneglycol 2000)-MPEG-2000-DSPE
1,2-distearoyl-sn-glycero-3-phosphoethanolamine, sodium salt

One source of phospholipid materials suitable for incorporation into the compositions of the invention is soy lecithin of high purity, i.e., free from allergenic, inflammatory agents or agents that cause other deleterious biological reactions, which is qualified for use in injectable products. Such injectable forms of soy lecithin are commercially available in the brand names of Phospholipon® by Phospholipid GmbH, Lipoid® S by Lipoid GmbH, Epikuron® by Degussa. These refined soy lecithin products may contain different concentrations of phosphotidylcholine (PC content) ranging from 30% to 100%. By combining lecithin products of different PC contents, it is possible to vary the consistency of the implant and persistence in the tissue.

Another source of phospholipids is the hydrogenated lecithin of soy or egg origins. Hydrogenation saturates the double bonds on the fatty acid side chains of the lecithin molecules. The resulted saturated fatty acids are less sensitive to the oxidation or enzymatic degradation. An implant comprising a hydrogenated lecithin is thus more stable chemically and degrades slower in the tissue than its naturally form. Examples of commercially available hydrogenated lecithin of injectable grade include Phospholipon® 90H, 100H by Phospholipid GmbH and LIPOID E PC-3 and LIPOID E PS-3, LIPOID S PC-3, LIPOID S PG-3, LIPOID S PA-3, and LIPOID S PE-3 from Lipoid GmbH.

The phospholipid component of the implant composition of the present invention is generally in the range of about 10% to about 90% of the total weight of the implant composition. In certain embodiments, the minimum range of the phospholipid component may be about 10%, 15%, 20%, 25%, 30%, 35%, 40%, 45%, 50%, 60%, 70%, 75%, 80%, or 85% (including any value between 10% and 75%). In certain embodiments, the maximum range of the phospholipid component may be about 40%, 45%, 50%, 60%, 70%, 75%, 80%, 85%, or 90% (including any value between 40% to 90%).

The “fluid carrier” is meant to be a pharmaceutically acceptable solvents or mixture thereof. Exemplary fluid carriers include, without limitation, water, an aqueous buffer solution (e.g., a phosphate buffered saline solution), ethanol, glycerol, propylene glycol, polyethylene glycol, vegetable oil, mono-, di- and triglycerides of long chain fatty acids (C12-C22) and mixtures thereof, mono-, di- and triglycerides of medium chain fatty acids (C6-C12) and mixtures thereof, mono-, di- and triglycerides of short chain fatty acids (C2-C6) and mixtures thereof, vitamin E and esters thereof, esters of fatty acids, ethyl oleate, n-methylpyrrolidone, glycofurol, 2-pyrrolidone, polyethylene glycol-15-hydroxystearate, polysorbates, polyoxyl castor oil, or combinations thereof.

For implant components that are sensitive to water, a non-aqueous fluid carrier comprising pharmaceutically acceptable vehicles can be used. An exemplary non-aqueous fluid carrier is a mixture comprising any one or more of glycerol, propylene glycol, ethyl oleate, ethanol and/or medium chain triglyceride. Such a fluid carrier is also preferred when the implant needs to be sterilized by filtration since the phospholipids can be dissolved at a moderately elevated temperature (about 60° C.) in said non-aqueous fluid carrier to form a clear solution, which can be filtered through a sterilizing filter of pore size rated at 0.2 micron.

Typically, a minimum amount of a non-aqueous fluid carrier is desired to minimize the potential tissue reaction to its components and to provide maximum volume of phospholipids in the implant for bulk volume and persistence in the host tissue. In certain embodiments, the volume of a fluid carrier need be sufficient to allow for reduction in particle size of phospholipids by homogenization such as via milling, sonication, mechanical agitation, high shear mixing, extrusion, microfluidization, heat treatment, etc. The reduction in particle size, in certain embodiments, results in syringeable or injectable implant compositions.

It is not necessary for the phospholipids to completely dissolve in the fluid carrier. Dispersion, such as an emulsion, a suspension, a paste, a gel (e.g., a hydrogel or an organogel), in particular, a gel or a paste, is suitable for the applications of this invention.

Alternatively, the fluid carrier may be added to a dry powder comprising pre-sized phospholipid particles just prior to implantation. In certain embodiments, an extemporaneous mixing of the fluid carrier and dry powder of phospholipid particles produces a syringeable (or injectable) paste. By adding the fluid carrier before the implantation, it allows for an improved stability of phospholipids and other components in the composition, which may be sensitive to components in the fluid carrier, in particular, water. For example, in a phospholipid containing PLGA or PLA polymer as the non-phospholipid filler material, it is preferred to mix the aqueous fluid carrier at the time of administration since the PLGA or PLA polymers are subject to hydrolysis in water.

As described above, the composition of the present invention may further comprise non-phospholipid filler components. “Non-phospholipid filler component” (also referred to as “non-phospholipid filler material”) refers to any substance that may be included in phospholipid compositions of the present invention other than the phospholipids, fluid carriers, or biologically active agents. Non-phospholipid filler components include biodegradable and non-biodegradable (permanent) materials. For compositions adapted for use as a tissue filler, non-phospholipid filler materials may be mixed with phospholipids to achieve desirable persistence, firmness, consistency, and/or injectability for a particular application. For instance, as facial anesthetics, it is desired that the dermal filler would last for no less than 6 months. The exemplary non-phospholipid filler materials are PMMA, PLA and PLGA. The exemplary concentration of PLA or PLGA in the implant may in the range of about 5% to about 50% (including any value therebetween) of the total weight of the implant. For compositions adapted for sustained local drug delivery, non-phospholipid filler components may regulate or modify, in combination with phospholipid components, the release rate or amount of biologically active agents that the compositions intend to deliver.

Non-phospholipid filler components useful in the present invention include, but are not limited to, (1) biodegradable and re-absorbable polymers (e.g., poly(DL-lactide-co-glycolide), poly(DL-lactide-co-glycolide)-COOH, poly(DL-lactide), poly(DL-lactide-COOH), poly(L-lactide), poly(glycolide), poly(e-caprolactone), poly(DL-lactide-co-caprolactone), poly(DL-lactide-co-caprolactone), and combinations thereof); (2) non-biodegradable polymers (e.g., polymethylmethacrylate, poly(vinyl alcohol) and copolymers thereof, sodium acrylate polymer, acrylamide polymer, acrylamide derivative polymer or copolymer, sodium acrylate and vinyl alcohol copolymer, vinyl acetate and acrylic acid ester copolymer, vinyl acetate and methyl maleate copolymer, isobutylene-maleic anhydride crosslinked copolymer, starch-acrylonitrile graft copolymer, crosslinked sodium polyacrylate polymer, crosslinked polyethylene oxide, and combinations thereof); (3) calcium phosphate minerals, hydroxyapatite, ceramics, titanium, or combinations thereof; (4) hydrogenated vegetable oil, glycerol esters of fatty acids, cholesterol, sodium cholesteryl sulfate, cholesterol derivatives, or combinations thereof; (5) polysaccharides (e.g., dextran, cyclodextrins, cellulose, sodium carboxymethylcellulose, agar methylcellulose, hydroxypropyl cellulose, hydroxypropyl methylcellulose, ethyl cellulose, microcrystalline cellulose, starch, amylose, amylopectin, pectin, alginates, chitin, chitosan, glycogen, hyaluronate, glycosaminoglycan, chondroitin, heparin, and combinations thereof; and (6) protein or amino acid polymers (e.g., collagen, gelatin, casein, albumin and combinations thereof).

In certain embodiments, the non-phospholipid filler component is present in the composition as fine particles, gels, or combinations thereof.

Various methods for incorporating non-phospholipid filler material in phospholipid compositions may be used. In certain embodiments, the non-phospholipid filler material may be mixed with the phospholipid component suspended in an aqueous fluid carrier. For the non-phospholipid filler material that is degradable in an aqueous environment, the resulting mixture may be further dried for storage and re-mixed with an aqueous fluid carrier to form an injectable gel of paste. In certain other embodiments, the non-phospholipid filler material and the phospholipid components may both be dissolved in a volatile organic solvent and then dried (directly or first forming an oil-in-water emulsion and then dried). The resulting dried material, if not already in fine powder, may be further micronized to fine powder. Dry particles selected for a particle size range (e.g., about 0.10 μm to about 200 μm, about 10 μm to about 100 μm, or about 20 μm to about 200 μm) may be subsequently suspended to form an injectable gel or a paste in a non-aqueous fluid carrier or in an aqueous fluid carrier just prior to injection.

One exemplary method to incorporate a permanent non-phospholipid filler material such as PMMA microspheres in a phospholipid composition of this invention is to mix the microspheres in the phospholipid component suspended in an aqueous fluid carrier.

One exemplary method to incorporate a biodegradable non-phospholipid filler material such as PLA or PLGA microspheres in a phospholipid composition of this invention is to mix the microspheres in the phospholipid component suspended in an aqueous fluid carrier and then subsequently remove the water by a conventional drying method such as vacuum drying, freeze-drying or spray drying.

An alternative exemplary method of incorporating the biodegradable PLA or PLGA in the phosphophlipid implant composition of this invention is to mix and dissolve both the PLA or PLGA and phospholipid materials in a volatile organic solvent such as methylene chloride to form a clear solution, the solution is then dried to completion to form a solid matrix which is subsequently micronized to fine powder. In certain embodiments, the fine powders selected in a size range from about 10 μm to about 100 μm in diameter are suspended to form an injectable paste in a non-aqueous fluid carrier or in an aqueous fluid carrier just prior to injection.

Yet another alternative exemplary method of incorporating the biodegradable PLA or PLGA in the phosphophlipid implant composition of this invention is to mix and dissolve both the PLA or PLGA and phsopholioid materials in a volatile organic solvent such as methylene chloride to form a clear solution, the solution is then added to an aqueous medium and is emulsified using a homogenizer to form an oil-in-water emulsion with the oil droplets of certain sizes. In certain embodiments, the emulsion is then freeze-dried or spray dried to obtain dry particles containing phosphoplids and PLA or PLGA. The particles selected in a size range from about 10 μm to about 100 μm in diameter are suspended to form an injectable paste in a non-aqueous fluid carrier or in an aqueous fluid carrier just prior to injection.

Yet another exemplary method of incorporating the biodegradable PLA or PLGA in the phospholipid composition of this invention is to mix and dissolve both the PLA or PLGA and phospholipid materials in a volatile organic solvent such as methylene chloride to form a clear solution, the solution is then spray dried to obtain dry particles containing phospholipids and PLA or PLGA. In certain embodiments, the particles selected in a size range from about 10 μm to about 100 μm in diameter are suspended to form an injectable paste in a non-aqueous fluid carrier or in an aqueous fluid carrier just prior to injection.

For the embodiments in which syringeable or injectable phospholipid compositions are provided, it is important that the total solid content and viscosity of the compositions be within a range which permits administration of the compositions through syringes, catheters, or needles such as those with a relatively narrow gauge, (e.g., 21 gauge, 22 gauge, or higher). For such embodiments, the total solid content, including phospholipids, non-phospholipid filler particles, and the like, will usually be in the range from about 10% (weight basis) to about 90%, usually being in the range from about 30% to about 70%, for example, being in the range from about 40% to about 60%. The corresponding viscosities will usually be in the range from about 0.4 Pa/sec to about 0.005 Pa/sec, usually being in the range from about 0.3 Pa/sec to about 0.05 Pa/sec, for example, being in the range from about 0.2 Pa/sec to about 0.1 Pa/sec.

In certain embodiments in which syringeable or injectable phospholipid compositions are provided, the majority of particles in the compositions are about 10 μm to about 200 μm, such as about 20 μm to about 200 μm, or about 10 μm to about 100 μm.

The compositions of the present invention may further include biocompatible fluid lubricants and/or viscosity modifiers, generally as described in U.S. Pat. No. 4,803,075, the disclosure of which is incorporated herein by reference. Exemplary lubricant components include glycerol, glycogen, maltose, and the like. Organic polymer base materials, such as polyethylene glycol and hyaluronic acid as well as non-fibrillar collagen, preferably succinylated collagen, may also act as lubricants. Such lubricants generally act to enhance the intrudability into soft tissue and improve the injectability by modifying the viscosity of the compositions.

The lubricant may be mixed first with one component of the composition (e.g., a fluid carrier) and then with other component(s) of the composition. Alternatively, it may be mixed with a mixture of more than one component of the composition (e.g., a mixture of a phospholipid component and a fluid carrier, or a mixture of a phospholipid component, a fluid carrier and a non-phospholipid filler component).

The compositions of the present invention, in certain embodiments may comprise one or more biologically active agents in a pharmaceutically effective concentration. Such biologically active agents may assist the use of the composition when used for tissue repair or augmentation. For instance, when used for hard tissue and bone implantation and repair, the compositions of the present invention may include additional components, such as osteogenic factors, as described generally in U.S. Pat. Nos. 4,888,366; 4,863,732; and 5,001,169, the disclosures of which are incorporated herein by reference. The compositions may also include autologous bone marrow, as generally described in U.S. Pat. No. 4,774,227, the disclosure of which is incorporated herein by reference. Alternatively, the biologically active agents are the substances to be locally deliveried via the phospholipid compositions. The presence of phospholipid and other components in the compositions allows for sustained release of the biologically active agents.

The biologically active agent may be mixed first with one component of the composition (e.g., a fluid carrier) and then with other component(s) of the composition. Alternatively, it may be mixed with a mixture of more than one component of the composition (e.g., a mixture of a phospholipid component and a fluid carrier, or a mixture of a phospholipid component, a fluid carrier and a non-phospholipid filler component). In certain embodiments, the biologically active agent need be dissolved in a solvent before being mixed with one or more of other components of the composition.

In certain embodiments, biologically active agents may be proteins and drugs, including tissue growth factors, such as FGF, PDGF, BMP, TGF-beta, and the like, which would promote healing and tissue repair at the site of administration.

Other exemplary biologically active agents that can be incorporated into the phospholipid compositions of the present invention include, but are not limited to, gene transfer vectors (e.g., DNA, RNA, plasmid DNA, DNA complexes, and viruses), local anesthetics, anti-inflammatory agents, anti-cancer or anti-tumor agents, anti-infectious agents, hormones, bone metabolism regulators, anti-convulsants, anti-depressants, analgesics, antipsychotic agents, anti-diabetic agent, anti-parkinisonian agents, smoking cessation aids, urinary tract agents, anti-osteoporosis agents, anti-obesity agents, fertility agents, contraceptives, and preservatives.

Exemplary anti-cancer or anti-tumor agents include, but are not limited to, 5-fluorouracil, anti-invasive factor, retinoic acid and derivatives thereof, platinum compounds, taxanes (e.g., paclitaxel), steroid derivatives, anti-metabolites, vinca alkaloids, adriamycin and doxarubicin, etoposide, arsenic derivatives, intercalating agents, alkylating agents (such as melphalan), and combinations thereof.

Exemplary local anesthetics include, but are not limited to, bupiricaine, procaine (novocaine), chloroprocaine (nesacaine), cocaine, lidocaine, tetracaine (amethocaine, pontocaine), mepivacaine, etidocaine (duranest), bupivacaine (marcaine), dibucaine (cinchocaine, nupercaine), prilocaine (citanest), benzoxinate (dorsacaine), proparacaine (alcaine, opthaine, opthetic), benzocaine (anesthesin), and butamben (butesin).

Exemplary anti-infectious agents (used interchangeably herein with “anti-infective agents”) include, but are not limited to, minocycline, bacitracin, polymyxin, neomycin, providone iodine, benzoyl peroxide, tolnaftate, miconazole, chlorhexidine, penicillin, oxacillin, clindamycin, carbenicillin, cephalosporins, ceforxitin, cefazolin, dicloxacillin, cloxacillin, and clavulanic acid, and mixture thereof.

In certain embodiments, the phospholipid composition of the present invention may further include cell adhesion promoters, such as endothelial-leukocyte adhesion molecule-1 (E-selectin or ELAM-1), vascular cell adhesion molecule-1 (VCAM-1), and intercellular adhesion molecule-1 (ICAM-1) and the like.

In certain embodiments, the phospholipid composition of the present invention may also include autologous cells. In certain other embodiments, the phospholipid compostion of the present invention may also include allogeneic or xenogeneic cells.

In certain embodiments, the compositions of the present invention are injectable, non-liposomal and in form of a gel or a paste. A “liposome” is a structure consisting of one or more concentric lipid bilayers separated by water or aqueous buffer compartments. These hollow structures, which have an internal aqueous compartment, can be prepared with diameters ranging from 20 nm to 10 μm. They are classified according to their final sizes and preparation methods as: small unilamellar vesicles (0.5-50 nm); large unilamellar vesicles (100 nm); reverse phase evaporation vesicles (0.5 μm), and large multilamellar vesicles (2-10 μm). A non-liposomal composition is a composition that does not contain a substantial amount (less than 5% (w/w)) of liposomes.

In certain embodiments, the phospholipid compositions are adapted for use as a tissue filler. A “tissue filler” (also referred to as “bulking agent”) is a composition that is implanted into a tissue to increase the volume of the tissue for cosmetic purposes or for treating disorders associated with an improperly reduced tissue volume. A tissue filler is generally biocompatible (i.e., substantially non-toxic), non-allergenic (i.e., produce no or tolerable levels of immune and inflammatory responses), and durable (i.e., present at the site of administration for at least one month). It may be biodegradable or partially biodegradable.

In certain embodiments, at least about 10%, 20%, 30%, 40%, or 50% of the phospholipid composition useful as a tissue filler according to the present invention is present at the site of administration at least 2, 3, 4, 5, 6, 7, 8, 9, 10, 11 or 12 months after its administration.

In certain embodiments, the phospholipid compositions of the present invention are adapted for sustained local drug delivery. Such compositions comprise (i) a phospholipid component in the range from about 10% to about 90% of the total weight of the composition), (ii) a pharmaceutically acceptable fluid carrier, and (iii) a biologically active agent in a pharmaceutically effective concentration.

“Sustained” refers to drug delivery where a composition that comprises a drug releases a pharmaceutically effective amount of the drug for at least one week. In certain embodiments, a pharmaceutically effective amount of a drug is released for at least 2, 3, 4, 5, 6, 7, 8, 9, or 10 weeks. In certain embodiments, the release rate is of zero order for at least one week.

In certain embodiments of phospholipid compositions useful for local drug delivery, the maximum amount of phospholipid components is at most about 15%, 20%, 25%, 30%, 35%, 40%, or 45%.

A composition comprises a biologically active agent in a “pharmaceutically effective concentration” if the composition releases a pharmaceutically effective amount of the biologically active agent.

Besides the phospholipid compositions described in the Examples section, additional exemplary phospholipid compositions include, but are not limited, the following compositions:

A composition comprising lecithin and phosphate-buffered physiological saline containing about 0.1% to about 1% (including any value therebetween, such as about 0.3%) lidocaine, wherein lecithin content is in the range from about 10% to about 90% of the total weight.

A composition comprising lecithin, polymethylmethacrylate microspheres and phosphate-buffered physiological saline containing about 0.1% to about 1% (including any value therebetween, such as about 0.3%) lidocaine, wherein lecithin content is in the range from about 10% to about 90% of the total weight of the composition, polymethylmethacrylate microspheres are in the range from about 5% to about 50% of the total weight of the composition.

A composition comprising lecithin, collagen and phosphate-buffered physiological saline containing about 0.1% to about 1% (including any value therebetween, such as about 0.3%) lidocaine, wherein lecithin content is in the range from about 10% to about 90% of the total weight of the composition, collagen is in the range from about 5% to about 20% of the total weight of the composition.

A composition comprising lecithin and particles of poly lactic acid polymer (PLA) or poly lactide-co-glycolide (PLGA) or a mixture thereof, wherein lecithin content is in the range from about 10% to about 90% of the total weight of the composition, and PLA or PLGA is in the range from about 5% to about 50% of the total weight of the composition.

A composition comprising lecithin, particles of poly lactic acid polymer (PLA) or poly lactide-co-glycolide (PLGA) or a mixture thereof, lidocaine and a fluid carrier selected from the group consisting of glycerol, propylene glycol, polyethylene glycol, ethyl oleate, medium chain triglyceride, and vegetable oil, wherein lecithin content is in the range from about 10% to about 90% of the total weight of the composition, PLA or PLGA is in the range from about 5% to about 50% of the total weight of the composition, lidocaine is at about 0.1% to about 1% (including any value therebetween, such as about 0.3%).

A composition comprising lecithin, lidocaine and a fluid carrier selected from the group consisting of glycerol, propylene glycol, polyethylene glycol of low molecular weight, ethyl oleate, medium chain triglyceride, vegetable oil and mixture thereof, wherein lecithin content is in the range from about 10% to about 90% of the total weight of the composition, lidocaine at about 0.1% to about 1% (including any value therebetween, such as about 0.3%).

A dry composition comprising lecithin and poly lactic acid polymer (PLA) or poly lactide-co-glycolide (PLGA) or a mixture thereof and a freeze-drying bulking agent selected from the group comprising poly alcohol, mono-, di-, oligo and poly-saccharides, amino acids, proteins and electrolytes, wherein lecithin content is in the range from about 10% to about 90% of the total weight of the composition, and PLA or PLGA is in the range from about 5% to about 50% of the total weight of the composition, and freeze-drying bulking agent in the range of about 10% to about 90% of the total weight. This composition is mixed by water to form a suspension or paste prior to implantation by injection.

A dry composition comprising lecithin and poly lactic acid polymer (PLA) or poly lactide-co-glycolide (PLGA) or a mixture thereof and a spray drying aid agent selected from the group comprising poly alcohol, mono-, di-, oligo and poly-saccharides, amino acids, proteins and electrolytes, wherein lecithin content is in the range from about 10% to about 90% of the total weight of the composition, and PLA or PLGA is in the range from about 5% to about 50% of the total weight of the composition, and freeze-drying bulking agent in the range of about 10% to about 90% of the total weight. This composition is mixed by water to form a suspension or paste prior to implantation by injection.

A composition comprising lecithin, a bone morphogenic protein and phosphate-buffered physiological saline, wherein lecithin content is in the range from about 10% to about 90% of the total weight, the bone morphogenic protein is in the range from about 0.1% to about 10% of the total weight.

A composition comprising lecithin, an antibiotic drug and a fluid carrier selected from the group consisting of glycerol, propylene glycol, polyethylene glycol of low molecular weight, ethyl oleate, medium chain triglyceride, vegetable oil and mixture thereof, wherein lecithin content is in the range from about 10% to about 90% of the total weight of the composition and the antibiotic drug is in the range at about 0.1% to about 10% of the total weight of the composition.

A composition comprising lecithin, a local anesthetic drug and a fluid carrier selected from the group consisting of glycerol, propylene glycol, polyethylene glycol of low molecular weight, ethyl oleate, medium chain triglyceride, vegetable oil and mixture thereof, wherein lecithin content is in the range from about 10% to about 90% of the total weight of the composition and the local anesthetic drug is in the range at about 0.1% to about 10% of the total weight of the composition.

A composition comprising lecithin, poly lactic acid polymer (PLA) or poly lactide-co-glycolide (PLGA) or a mixture thereof and an anticancer drug and a fluid carrier selected from the group consisting of glycerol, propylene glycol, polyethylene glycol of low molecular weight, ethyl oleate, medium chain triglyceride, vegetable oil and mixture thereof, wherein lecithin content is in the range from about 10% to about 90% of the total weight of the composition and the anticancer drug is in the range at about 0.1% to about 10% of the total weight of the composition.

The components of the phospholipid material of the present invention may be combined and/or processed in any manner that provides for a substantially homogeneous mixture. For example, components may be mixed homogeneously by repeated passage through pumps or repeated transfer between adjacent syringes having a small diameter interconnecting channel. A suitable syringe device providing the necessary mixing is described in U.S. Pat. No. 4,743,229, the disclosure of which is incorporated herein by reference. In addition, in certain embodiments, the resulting mixture may be mechanically agitated to reduce the size of microparticles to produce syringeable (or injectable) implant compositions. Mixing various components of the phospholipid compositions may be performed prior to the administration of the compositions into an animal (e.g., human) or at the site of implantation.

The phospholipid compositions of the present invention may be administered intradermally or subcutaneously into humans or other mammals to augment soft tissue, to repair tissue defects, to correct congenital anomalies, to correct cosmetic defects, and the like. Such defects or anomalies may be caused by aging, environmental exposure, weight loss, child bearing, surgery, diseases (e.g., acne and skin cancer), or combinations thereof. The defects or anomalies include, but are not limited to, frown lines, worry lines, wrinkles, crow's feet, marionette lines, stretch marks, and internal or external scars resulted from injury, wound, surgery, bites, cuts, or accidents. The compositions of the present invention may also be injected into internal tissues to augment such tissues or treating diseases. For instance, the compositions of the present invention may be injected into the vocal cord, nose, and the tissues defining body sphincters (e.g., the lower esophageal sphincter, the diaphragm, the bladder sphincter or urethra) for augmenting or repairing such tissues and treating diseases such as gastroesophageal reflux disease, urinary incontinence (e.g., caused by bladder-neck hypermobility) or urinary reflux disease.

The phospholipid compositions of the present invention may also be used for repair or augmentation of hard tissues, such as bone, cartilage, connective tissues, and the like. Hard tissue and bone augmentation and repair are described generally in U.S. Pat. Nos. 5,001,169; 4,863,732; 4,563,350, the disclosures of which are incorporated herein by reference.

The phospholipid compositions of the present invention may also be used in local delivery of a biologically active agent. Such delivery may be used for treating a solid tumor where the biologically active agent is an anti-cancer agent, for treating chronic pain where the biologically active agent is an anesthetic, or treating chronic periodontal disease where the biologically active agent is an anti-infectious agent.

The phospholipid compositions of the present invention may be administered by any appropriate methods in the art. For instance, the compositions may be administered through an incision at the site of implantation. In certain embodiments, the compositions may be administered into a subject via a syringe, a catheter, or injected using a needle (e.g., those with 21 gauge or higher).

The compositions of the present invention may be stored as a kit, where the separate individual components (i.e., the phospholipid component, the fluid carrier, and other optional components) are packaged separately or as a mixture. The kit may further comprise instructions for making the phospholipid compositions (if the individual components are packaged separately) and for using the phospholipid compositions. For instance, in certain embodiment, the present application provides a kit for preparing an injectable non-liposomal composition in form of a gel or a paste that comprises a container containing one or more phospholipids, another container containing a pharmaceutically acceptable fluid carrier, and instructions for mixing the phospholipid(s) and the pharmaceutically acceptable fluid carrier to produce an injectable non-liposomal composition in form of a gel or a paste.

The following examples are offered by way of illustration, not by way of limitation.

### EXAMPLES

### Example 1

### Preparation of Phospholipid Pastes in Non-Aqueous Fluid Carriers and In Vivo Evaluation for Biocompatibility in Human

Two uniform lecithin gel/pastes were prepared to contain the following components:

| | % w/w | |
|---|---|---|
| Component | F-2 | F-3 |
| Soy lecithin (Phospholipon ® 90G) | 15.9 | 32.7 |
| Medium chain triglyceride (Miglyol ® 812) | 15.9 | 12.7 |
| Sucrose, NF | 15.9 | 12.7 |
| Ethanol, USP | 4.5 | 3.6 |
| Propylene glycol, USP | 47.8 | 38.3 |
| Total | 100 | 100 |

Weigh out and combine soy lecithin (Phospholipon® 90G, an injectable grade soy lecithin containing about 90% phosphotidylcholine by Phospholipid GmbH), medium chain triglyceride (Miglyol® 812 by Sasol Corp.), sucrose, NF and propylene glycol, USP in a clean container, add anhydrous ethanol, USP to dissolve all solids to form a clear and yellow solution. Apply vacuum to remove ethanol until the residual ethanol content is less than 5% of the total weight. Warm up the mixture to 60° C. to form a transparent solution and then filter the solution through a sterilizing filter. Cool down the filtrate to room temperature to obtain a yellow, translucent and uniform gel.

The resulting formulation (F-3) was self-injected subdermally at 0.1 ml volume into the skin of a forearm of a human volunteer (a cosmetic surgeon). It caused some swelling but no pain, and was palpable after 7 days. This formulation was rated as very well biocompatible.

### Example 2

### Preparation of Phospholipid Pastes Imbedded with PMMA Microspheres in a Non-Aqueous Fluid Carrier and In Vivo Evaluation for Biocompatibility in Human

Uniform lecithin pastes was prepared to contain the following components:

| | % w/w | |
|---|---|---|
| Component | F-4 | F-5 |
| Hydrogenated soy lecithin (Phospholipon ® 90H) | 33.4 | 30 |
| PMMA microspheres | | 20 |
| Propylene glycol, USP | 33.3 | 25 |
| Ethyl oleate, EP | 33.3 | 25 |
| Total | 100 | 100 |

Weigh out and combine hydrogenated soy lecithin (Phospholipon® 90H, an injectable grade soy lecithin containing about 90% hydrogenated phosphotidylcholine by Phospholipid GmbH), propylene glycol (USP) and ethyl oleate (Crodamol EO by Croda) in a clean container, and heat the mixture to about 60° C. to obtain a transparent and slightly yellow solution. Filter the solution through a 0.2 micron sterilizing filter into sterile syringes. Place the syringes in an autoclave bag, and terminally sterilize the syringes and the contents therein using a 60-minute autoclave cycle (250° F.). Cool down the contents in the syringes to room temperature to obtain a thick, opaque, off-white and uniform paste (F-4).

For F-5, combine 20 parts by weight of PMMA microspheres (PMMA-B344, 40.17 μm±0.76 μm, by Microparticles GmbH, Berlin) and 80 parts by weight of F-4, heat the mixute up to 60° C. to liquefy F-4 paste and then mix it well with the PMMA microspheres. Fill the mixture into sterile syringes and terminally sterilize the syringes and the contents therein using a 60-minute autoclave cycle (250° F.). Cool down the contents in the syringes to room temperature to obtain a thick, opaque, off-white and uniform paste (F-5).

Clinical observations The F-5 formulation was self-injected subdermally at as 3×0.1 ml blebs into the skin of a forearm of a human volunteer (a cosmetic surgeon). There was a burning sensation of about 10 seconds, which could be diminished with an addition of 0.3% lidocain (as in the collagen or Artefill filler products). The subsequent swelling (edema) was seen for 2 days, lessened to day 4 and was still recognizable at day 10 (Erythema, bruising and swelling are typical adverse events for dermal filler products, such as Hylaform®, Cosmoderm, CosmoPlast, Zyderm and Zyplast). After about 1 month, the upper bleb was excised for histology examination. After 3 months, the 2 remaining implants were still palpable.

Histology After about 1 month, the upper bleb was excised and the samples were fixed and stained with Masson Trichrome for examination under the microscope. The implant at 1 month showed no foreign body giant cells, and no signs of inflammatory cells in the surrounding of the implant.

The F-5 formulation was concluded as non-sensitizing, non-irritating and was well tolerated by an expert of cosmetic surgery and aesthesia. The phospholipid compositions of this example can be used as a biocompatible vehicle/co-implant material for PMMA microspheres.

### Example 3

### Preparation of a Phospholipid Paste in an Aqueous Fluid Carrier

A uniform lecithin paste was prepared to contain the following components:

| | % w/w |
|---|---|
| Component | F-6 |
| Hydrogenated soy lecithin (Phospholipon ® 90H) | 30 |
| Purified water | 70 |
| Total | 100 |

Weigh out and combine 15 parts by weight of hydrogenated soy lecithin (Phospholipon® 90H, an injectable grade soy lecithin containing about 90% hydrogenated phosphotidylcholine by Phospholipid GmbH) and 75 parts by weight of purified water in a clean container, heat the resulting mixture to about 60° C. and agitate it vigorously until a uniform paste is obtained. Apply vacuum to the paste to remove water until the water content is 70% w/w. Fill the paste into sterile syringes. Place the syringes in an autoclave bag, and terminally sterilize the syringes and their contents using a 60-minute autoclave cycle (250° F.). Cool down the contents in the syringes to room temperature to obtain a thick, opaque, off-while and uniform paste (F-6).

### Example 4

### Preparation of a Phospholipid Paste Containing Biodegradable PLA

A uniform phospholipid paste was prepared to contain the following components:

| | % w/w |
|---|---|
| Component | F-7 |
| Hydrogenated soy lecithin (Phospholipon ® 90H) | 7.14 |
| PLA (Absorbable Polymers International) | 7.14 |
| Glycerin | 85.71 |
| Total | 100 |

Weigh out and combine by weight of hydrogenated soy lecithin (Phospholipon® 90H, an injectable grade soy lecithin containing about 90% hydrogenated phosphotidylcholine by Phospholipid GmbH) and PLA (Absorbable Polymers International) in a clean container, add ethyl acetate, mix and heat to 90-100° C. briefly until all solids are dissolved. Add glycerin (Dow chemical) and deionized water and mix vigorously to form a crude emulsion. Pass the crude emulsion through a high-pressure homogenizer (Microfluidizer 110L by microfluidics) to obtain a fine emulsion. Fill the fine emulsion into a clean vial and freeze-dry to remove ethyl acetate and water and to obtain a soft phospholipid-PLA paste. The paste was injectable through a 28G needle.

This paste is intended as an injectable and biodegradable filler with a sustained filling action last for at least 6 months since the PLA used in this paste is known to have an in vivo residence time of 6-12 months.

### Example 5

### Preparation of Phospholipid Paste Containing a Drug Minocycline

A uniform phospholipid paste can be prepared to contain the following components using a similar procedure as described in EXAMPLE 2.

| Component | % w/w |
|---|---|
| Hydrogenated soy lecithin (Phospholipon ® 90H) | 30 |
| Minocycline | 0.2 |
| Propylene glycol, USP | 25 |
| Ethyl oleate, EP | 25 |
| Total | 100 |

This drug-containing phosphoplipid paste is intended as a subgingival filler to be applied to the periodontal pocket. The soft paste allows easy delivery of an accurate dose by extrusion through a syringe and cannula. Once placed in the periodontal pocket, propyelene glycol/ethayl oleate would quickly diffuse away, resulting in a harden matrix of phospholipid in which minocycline is incorporated. The release of minocycline from the matrix would be controlled by its slow diffusion from the matrix and the erosion of the matrix. A slow release of minocycline is thus accomplished.

This drug-containing phosphoplipid paste may be used for scaling and root planning procedures for reduction of pocket depth in patients with adult periodontitis. It may be used as part of a periodontal maintenance program that includes Mucous Membrane Disorder.

### Example 6

### Preparation of Phospholipid Paste Containing Another Drug Bupiricaine

A uniform phospholipid paste can be prepared to contain the following components using a similar procedure as described in EXAMPLE 2.

| Component | % w/w |
|---|---|
| Hydrogenated soy lecithin (Phospholipon ® 90H) | 30 |
| Bupiricaine HCl | 2 |
| Propylene glycol, USP | 25 |
| Ethyl oleate, EP | 25 |
| Total | 100 |

This drug-containing phosphoplipid paste can provide an ultralong-acting local anesthetic that would benefit patients with acute and chronic pain, while currently available local anesthetics have relatively brief durations of action. It may be administered by intradermal or intramuscular injections to patients with chronic al pain such as back pain.

### Example 7

### Preparation of Phospholipid Paste Containing Another Drug 5-Fluorouracil (5-FU)

A uniform phospholipid paste can be prepared to contain the following components using a similar procedure as described in EXAMPLE 4.

| | % w/w |
|---|---|
| Component | F-7 |
| 5-Fluorouracil (5-FU) | 5-10 |
| Hydrogenated soy lecithin (Phospholipon ® 90H) | 7.14 |
| PLA (Absorbable Polymers International) | 7.14 |
| Glycerin | 85.71 |
| Total | 100 |

This drug-containing phospholipid paste can be injected directly into solid tumors (i.e., intratumor injection) to achieve a high concentration of 5-Fluorouracil in the tumor tissues while maintaining a low drug concentration in the healthy tissues surrounding the tumor. The phospholipids and PLA would provide a sustained release of 5-Fluorouracil allowing a prolonged action of the anticancer agent. This phospholipid injectable composition may be used to treat various solid tumors such as head and neck cancers, gastric cancers, lung cancers, and liver cancers, etc.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An injectable non-liposomal composition adapted for use as a tissue filler in form of a gel or a paste comprising a phospholipid component and a pharmaceutically acceptable fluid carrier, wherein the phospholipid component is in the range from about 10% to about 90% of the total weight of the composition.

2. The composition according to claim 1, wherein the pharmaceutically acceptable fluid carrier is selected from the group consisting of water, an aqueous buffer solution, ethanol, glycerol, propylene glycol, polyethylene glycol, vegetable oil, mono-, di- and triglycerides of long chain fatty acids (C12-C22) and mixtures thereof, mono-, di- and triglycerides of medium chain fatty acids (C6-C12) and mixtures thereof, mono-, di- and triglycerides of short chain fatty acids (C2-C6) and mixtures thereof, vitamin E and esters thereof, esters of fatty acids, ethyl oleate, n-methylpyrrolidone, glycofurol, 2-pyrrolidone, polyethylene glycol-15-hydroxystearate, polysorbates, polyoxyl castor oil and combinations thereof.

3. The composition according to claim 1, wherein the phospholipid component is selected from the group consisting of naturally occurring phospholipids and synthetic phospholipids.

4. The composition according to claim 3, wherein the naturally occurring phospholipids is selected from the group consisting of soy lecithin, egg lecithin, hydrogenated soy lecithin, hydrogenated egg lecithin, sphingosine, gangliosides, and phytosphingosine and combinations thereof.

5. The composition according to claim 3, wherein the synthetic phospholipid is selected from the group consisting of diacylglycerols, phosphatidic acids, phosphocholines, phosphoethanolamines, phosphoglycerols, phosphoserines, mixed chain phospholipids, lysophospholipids, pegylated phospholipids and combinations thereof.

6. The composition according to claim 1 further comprising a non-phospholipid filler component.

7. The composition according to claim 6, wherein the non-phospholipid filler component is selected from the group consisting of poly(lactide-co-glycolide), poly(lactide-co-glycolide)-COOH, poly(lactide), poly(lactide-COOH), poly(lactide), poly(glycolide), poly(e-caprolactone), poly(lactide-co-caprolactone), poly(lactide-co-caprolactone), polymethylmethacrylate, poly(vinyl alcohol) and copolymers thereof, sodium acrylate polymer, acrylamide polymer, acrylamide derivative polymer or copolymer, sodium acrylate and vinyl alcohol copolymer, vinyl acetate and acrylic acid ester copolymer, vinyl acetate and methyl maleate copolymer, isobutylene-maleic anhydride crosslinked copolymer, starch-acrylonitrile graft copolymer, crosslinked sodium polyacrylate polymer, crosslinked polyethylene oxide, calcium phosphate minerals, hydroxyapatite, ceramics, titanium, hydrogenated vegetable oil, glycerol esters of fatty acids, cholesterol, sodium cholesteryl sulfate, cholesterol derivatives, dextran, cyclodextrins, cellulose, sodium carboxymethylcellulose, agar methylcellulose, hydroxypropyl cellulose, hydroxypropyl methylcellulose, ethyl cellulose, microcrystalline cellulose, starch, amylose, amylopectin, pectin, alginates, chitin, chitosan, glycogen, hyaluronate, glycosaminoglycan, chondroitin, heparin, a protein or polymer of amino acids selected from the group consisting of collagen, gelatin, casein, albumin and combinations thereof.

8. The composition according to claim 1 further comprising at least one biologically active agent.

9. The composition according to claim 8, wherein the biologically active agent is selected from the group consisting of tissue growth factors, osteogenic factors, hormones, and bone marrow.

10. The composition according to claim 8, wherein the biologically active agent is selected from the group consisting of gene transfer vectors, local anesthetics, anti-inflammatory agents, anti-cancer agents, anti-infectious agents, hormones, bone metabolism regulators, anti-convulsants, anti-depressants, analgesics, antipsychotic agents, anti-diabetic agents, antiparkinisonian agents, smoking cessation aids, urinary tract agents, anti-osteoporosis agents, anti-obesity agents, cardiotonic agent, fertility agents, contraceptives, preservatives, and cell adhesion promoters.

11. The composition according to claim 1 further comprising a biocompatible fluid lubricant.

12. A method for repairing or augmenting a tissue comprising administering the composition according to claim 1 into a mammal in need thereof.

13. The method according to claim 12, wherein the tissue is a hard tissue or a soft tissue.

14. The method according to claim 12 wherein the tissue is a dermal tissue.

15. The method according to claim 13 wherein the augmenting is to treat frown lines, worry lines, wrinkles, crow's feet, marionette lines, stretch marks, internal or external scars resulted from injuries, wounds, surgeries, bites, cuts, or accidents, acne, skin cancer, vocal cord disorders, gastroesophageal reflux disease, urinary incontinence, or urinary reflux disease.

16. A method of local delivery of a biologically active agent comprising administering the composition according to claim 8 in a patient in need thereof.

17. The method according to claim 16 wherein the local delivery of a biologically active agent is to treat cancer, chronic pain, or a chronic periodontal disease.

18. A method for preparing an injectable, non-liposomal composition adapted for use as a tissue filler in form of a gel or a paste, comprising providing a mixture that comprises one or more phospholipids with a pharmaceutically acceptable fluid carrier, wherein the phospholipid(s) are in the range from about 10% to about 90% of the total weight of the mixture, and homogenizing the mixture to produce an injectable, non-liposomal composition adapted for use as a tissue filler in form of a gel or a paste.

19. The method according to claim 18 wherein further comprising sterilizing the composition by filtration, heat, radiation, electron beam, or a combination thereof.

20. A kit comprising the composition according to claim 1 and instructions for using the composition.
